(19) United States
(12) Patent Application Publication
(10) Pub. No.: US 2013/0203864 A1
(43) Pub. Date: Aug. 8, 2013

(54) Method of Production of Cosmetics Emulsion

(71) Applicant: Andrej Getalov, Moscow (RU)

(72) Inventor: Andrej Getalov, Moscow (RU)

(21) Appl. No.: 13/819,495

(22) PCT Filed: Aug. 10, 2011
(86) PCT No.: PCT/RU2011/000603

§ 371 Date: Mar. 25, 2013

(30) Foreign Application Priority Data
Sep. 8, 2010 (RU) 2010137176

(51) Int. Cl. A61K 8/06; A61Q 19/00

(52) U.S. Cl. CPC A61K 8/06; A61Q 19/00
USPC 514/772.3

(57) ABSTRACT

The invention relates to the field of cosmetology and is referred to the technology for obtaining cosmetic skin care products. A method of manufacturing of cosmetic products includes gradual dispersion of components at room temperature at that the components are added to the plant with a powerful hydroacoustic effect, where dispersion of components and cavitation homogenization of emulsion with the further filling are made, at that the mode of acoustic cavitation is generated due to the double resonance effect in a flow channel. Mechanical oscillation of the rectangular flow channel system generates sound waves, forming of a standing wave at the fundamental frequency for the wall or walls of the flow channel. These standing waves in turn form a quasi-plane standing wave in a moving multi-phase media consisting of mixed ingredients. The method obtains superfine monodispersed cream emulsions with submicron size of the dispersed phase, resistant to coalescence, which significantly improve the organoleptic characteristics of cosmetic products, to allow reducing significantly the volume of ingredients required in the process of production of an emulsifier.

### CROSS REFERENCE TO RELATED APPLICATION

This application is for entry into the U.S. National Phase under §371 for International Application No. PCT/RU2011/000603 having an international filing date of Aug. 10, 2011, and from which priority is claimed under all applicable sections of Title 35 of the United States Code including, but not limited to, Sections 120, 363 and 365(c), and which in turn claims priority to Russian National Application RU 2010137176 filed on Sep. 8, 2010.

### BACKGROUND

1. Field of the Invention

The invention relates to the field of cosmetology and dermatology, and can be used in biology, pharmacology, cosmetics industry, veterinary and food industry, in particular in cosmetics industry—while development of technologies for obtaining cosmetic products for skin, hair and nails care.

2. Description of Related Art

It is known that the penetration of biologically active substances into the deeper tissues of the skin also depends on the dimension and homogeneity of the oil phase of the cosmetic cream, which includes vegetable and essential oils, a number of important extracts and other fat-soluble ingredients. As a rule, in the technology of the cream it tends to break up the oil phase droplets as small as possible.

In this case, along with liposomes of “oil in water” emulsion, biologically active compounds can penetrate through skin tissues being dissolved in the oil phase of the emulsion and adsorbed at the structural interface.

There is a method of production of cosmetic cream currently in use that includes the following stages of technological process:

Weighing and melting of the raw materials;

Preparation of fatty and aqueous phase;

Emulsification;

Cooling and perfume;

Packing in the packing materials. For the preparation of the aqueous phase the ingredients are heated to 75-80° C. To prepare the oil phase the ingredients are heated to 80-85° C. Further, the fat and aqueous phases are mixed. Under certain conditions (temperature, pH environment, incorporation orde) DNA and preservatives are added to a creamy mass.

The disadvantage of this method is the considerable power capacity of the cream production technologies and reducing of the biological activity of its components during the preparation of the product due to the fact that the production process of the emulsion is made by heating to 80-85° C. Further, there is homogenization of the two phases, which makes it more difficult for the input of components and additives being critical to thermal decomposition (temperature up to 40-45° C.) and at the same time being required for homogenization. These weak points significantly limit the application area of the method, in particular for the preparation of emulsions (creams, lotions, etc.) with the presence of natural vitamin supplements.

There is a method of production of cosmetic emulsion currently in use comprised of preparing basis by dispersing the vegetable oil, emulsifier and glycerol in the dispersion medium, followed by adding to the emulsion in the process of mixing of the biologically active substances of plant and animal origin. The emulsifier polyethylene oxide gel is used, and dispersion is carried out at room temperature.

The disadvantage of this analog method is the fact that for the intensification of the processes of dissolution and dispersion, as well as for preparation fine emulsions and suspensions the volumes are additionally equipped with high-speed stirring devices. Another disadvantage of this method is the inability of the homogenization process (cold emulsification) while the incorporation of biologically active substances into the cream to produce emulsions with submicron particle size.

The closest to the subject invention method is the method of manufacturing cosmetic products in the form of an emulsion comprising a dispersion of partially soluble components, emulsifiers, biologically active substances (having any origin), and solid powdered ingredients (sorbents or abrasives) in the solvent at room temperature where the components are added simultaneously or sequentially through individual dosing plants directly into the insonation camera of rotary-cavitation plant where cavitation emulsification process is implemented with simultaneous passing through the insonation chamber of emulsion complex “Mirra” (or any other) and an aqueous solution.

The nature of the subject invention simmers down to the use of cavitation homogenization (emulsification) concept to obtain highly efficient organic cosmetic and active cosmetic emulsion funds.

The application of rotary-cavitation treatment method for manufacturing cosmetic emulsions has several advantages over traditional methods of emulsification.

However, the process, or rather the mode of rotary-homogenizing into the insonation chamber of rotary machines, requires more specification.

It is known that in practice in the insonation chamber and in the channels of the stator of rotary rig different modes of cavitation of acoustic waves can be implemented.

Such wordings as “rotary cavitation plant with power hydroacoustic impact” and “mixture obtained in rotary cavitation plant by the cavitation emulsification method” should be specified. The question is only about the optimal selection of operating modes of rotary cavitation plants, as they depend on many factors.

For example, the device technically implements cavitation mode in almost all the differential pressure, but it may occur as hydrodynamic cavitation mode and acoustic cavitation mode. And the peculiarities of resonance phenomena in rotary machines also include the existence of multiple resonances and their complex interdependence with the design parameters of the plant, operation modes and the median characteristic. Each structural component is the generator of the particular frequency spectrum, which eventually superpose (superposition) to each other. The process of dissolution of sulfur in the oil mixture is optimum while implementation of the operation mode of rotary machine, exactly when it is excited by an acoustic pulse cavitation. There were devised a number of constructional decisions of rotor-stator pair and pipes nozzles for setting a resonant superposition of the frequencies for a particular technological process.

One of the authors of the prototype method in an interview suggests that the frequency of exposure while rotary cavitation homogenization is 3 times the frequency of the current in the power supply, i.e., is about 150-180 Hz. According to the results, this is probably one of the resonant modes, and there are resonances at higher frequencies with greater spectral density and a better Q factor. In particular, there are registered resonant frequencies˜540-580 Hz.

Thus, we can note the following significant disadvantages of the prototype:

Difficult choices of the optimum operation mode of the rotary cavitation plant to obtain the required particle size and homogeneity of the final emulsion;

The upper limit on the resonant frequency, which in practice will not exceed˜2000 Hz, due to the complex structure with many parts, each of which is the vibration transducer, which eventually superpose to each other and are not always in phase;

In practice, it is known that after passing through the homogenizer in the emulsion there is initiated a process of coalescence (interflowing) of droplets of the dispersed phase, even if the initial droplet size is small, dozens of nanometers and finally a stable emulsion is obtained, but with a wide range of size distribution of the dispersed phase size.

### SUMMARY

In one aspect, a method of production of cosmetics emulsion is provided. The invention relates to the field of cosmetology and is referred to the technology for obtaining cosmetic skin care products. The method contemplated herein of manufacturing of cosmetic products includes gradual dispersion of components at room temperature at that the components are added to the plant with a powerful hydroacoustic effect, where dispersion of components and cavitation homogenization of emulsion with the further filling are made, at that the mode of acoustic cavitation is generated due to the double resonance effect in the flow mechanical oscillation rectangular channel system of finite length, on opposite sides of the channel the generating of sound waves with forming of a standing wave at the fundamental frequency for the given wall of the channel is inphase provided, which in turn form a quasi-plane standing wave in a moving multi-phase media consisting of mixed ingredients, at that the gap width h of the channel is chosen multiple of quarter wavelength, excited in the multiphase medium by the channel walls

h=(k/4)*(C/f), k=1,2,3, . . .
where
f—the frequency of fundamental harmonic of the standing wave of the channel wall, Hertz;
C—the sound velocity in a multiphase medium, meter per second;
h—the distance between the walls of the channel, meters;
   and the amplitude of oscillation of the channel wall, is sorted out optimal for the different phases of manufacturing the emulsion is above the threshold of acoustic cavitation for the particular processed moving multiphase medium.
   The method takes an opportunity to obtain superfine monodispersed cream emulsions with submicron size of the dispersed phase, resistant to coalescence, which significantly improve the organoleptic characteristics of cosmetic products, allow reducing significantly the volume of ingredients required in the process of production of an emulsifier.

### BRIEF DESCRIPTION OF THE DRAWINGS

FIG. 1 provides a view of an embodiment of nutritional cream with healing herbs.

FIG. 2 provides a view of an embodiment of a calibration scale.

FIG. 3 provides a chart of a size distribution of a dispersed phase of an embodiment of a cream.

FIG. 4 provides a view of an embodiment of an apparatus to create the cosmetics emulsion.

FIG. 5 provides a view of an industrial plant embodiment of a system to create the cosmetics emulsion.

FIG. 6 provides a chart of a spectrum of vibrations experienced by the cosmetics emulsion during treatment.

FIG. 7 provides a view of an embodiment of the nodal lines and loops.

FIG. 8 provides a comparison of creams on the same magnification scale.

FIG. 9 provides a chart of a size distribution of a dispersed phase of the cosmetics emulsion.

### DETAILED DESCRIPTION

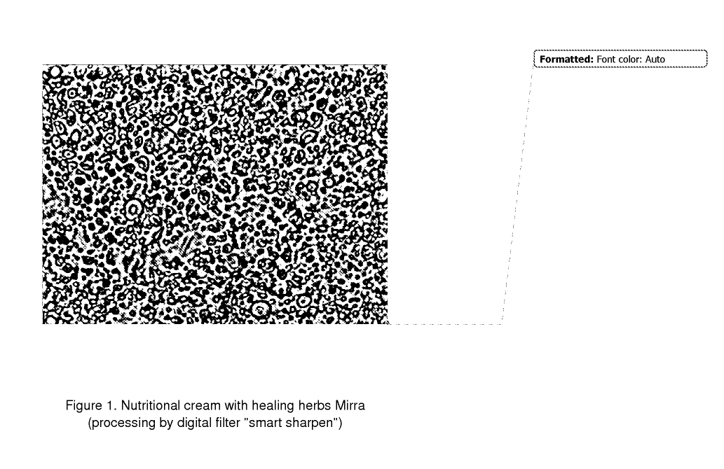

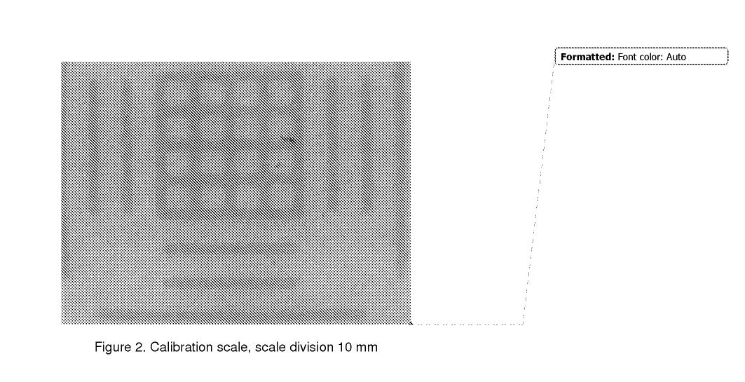

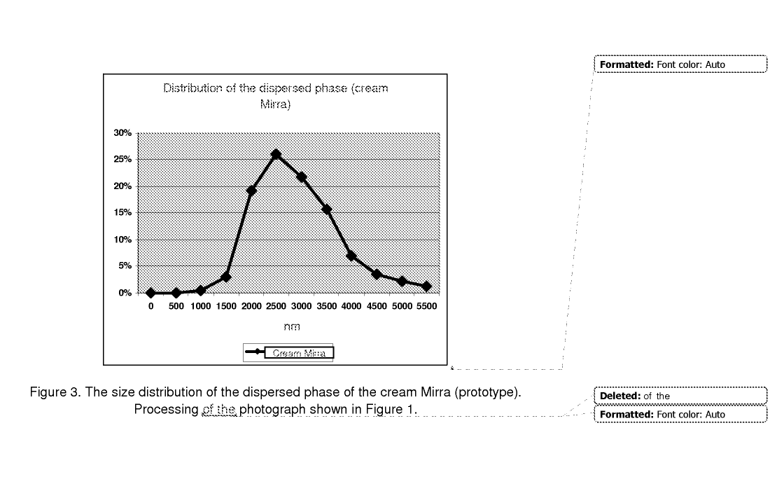

FIG. 1 represents a photograph of cream produced by JSC Mirra-M (Nutritional cream with healing herbs) photograph after processing digital filter “smart sharpen” obtained with an optical microscope with an increase in 1000 and a digital packed with 5.2 megapixel resolution. To estimate the size of the dispersed phase object-micrometer FIG. 2 with a scale 10 mm was used. For digital image processing the application Image Scope was used. The differential size distribution of the dispersed phase (processing results) is presented in FIG. 3. As it can be seen from the obtained results used by Mirra Company, rotor—cavitation homogenizers cannot provide long-term stable existence of the dispersed phase size of˜500-600 nm, which is considered close to the optimum for human skin, although immediately after completing of the homogenization process the share of this phase may be higher.

The research carried out by the company “DERMANIKA” of over 40 different creams (with and without emulsions) of leading manufacturers have shown that the size distribution of the dispersed phase is close to each of the products of all these manufacturers (the difference is not more than 2 times of the size of the fundamental mode) and is determined by the applied types of rotary homogenizers.

In addition to the undertaken research, the optimal power and frequency characteristics of acoustic waves was determined for the treated emulsion to produce significantly better performance characteristics on the size of the dispersed phase and the stability of the emulsion, to obtain correlations between the size of the dispersed phase and the frequency and level of homogeneity and Q-factor characteristic of oscillation channel system. There were found the effective methods of reducing peroxide value (specifies the number of free radicals) to the regulatory requirements.

The above research has been partially reported at the XIV International Research and Practice conference “Cosmetics and raw materials: safety and efficiency” in October 2009, where they were awarded second place and a diploma, there are publications in the specialized magazines. There were implemented resonant modes being close to plane (quasi-plane) standing acoustic wave in the flowing emulsion in rectangular channel (the channel length is much more than the distance between the side walls).

In this case, in accordance with the criteria (the threshold) of cavitation and the operation in resonant mode with maximum efficiency better characteristics for the intensification of combined physical-chemical, hydromechanical, heat-exchanging and mass-exchanging processes in the processing medium as well as the minimum size and homogeneity of fat (oil) phase obtained on output.

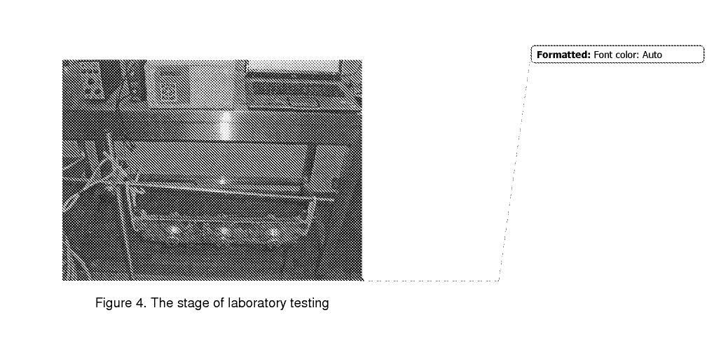

This technology may be implemented on an industrial scale in the operating cosmetic production plant “CJSC EMANSI Laboratory.” The photograph of the industrial plant is represented in FIG. 4. The first products manufactured by this technology, hand cream Anti Smell Smoke (for smokers, against the influence of nicotine and smoke to hand skin), took the whole cycle of certification tests (Sanitary and epidemiological inspection report No. 77.01.12.915.P.006156.02.10 of Feb. 3, 2010 and the Declaration of conformity, the results of which are also confirmed by independent testing of laboratory “Spectrum”, accreditation certificate No. ROSS RU.0001.21PSH50) with the corresponding protocol No. 19 dated Dec. 22, 2009.)

The aim of the present invention is to reduce the average size of the dispersed phase wile obtaining any kind of emulsion (with or without) and improvement of the homogeneity (dimensional homogeneity).

This goal is achieved by the fact that the regime of quasi-plane resonant wave and acoustic cavitation is generated due to the double resonance effect in a flow channel, and mechanical oscillation of the rectangular flow channel system of finite length. On opposite sides of the flow channel sound waves are generated, and are selected to form a standing wave at the fundamental frequency for the given wall of the channel. These generated sound waves in turn form a quasi-plane standing wave in a multi-phase mixture moving through the flow channel. The mixture consisting of mixed ingredients medium in the gap between the channel walls, at that the gap width of the channel is chosen multiple of quarter wavelength, excited in the multiphase medium by the channel walls:

h=(k/4)*(C/f), k=1,2,3, . . .
where
f—the frequency of fundamental harmonic of the standing wave of the channel wall, Hertz;
C—the sound velocity in a multiphase medium, meter per second;
h—the distance between the walls of the channel, meters;
   and the amplitude of oscillation of the channel wall is sorted out optimal for the different phases of manufacturing the emulsion is above the threshold of acoustic cavitation for the particular processed moving multiphase medium.

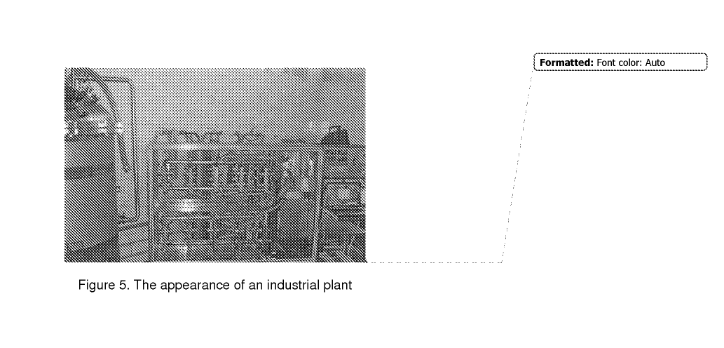

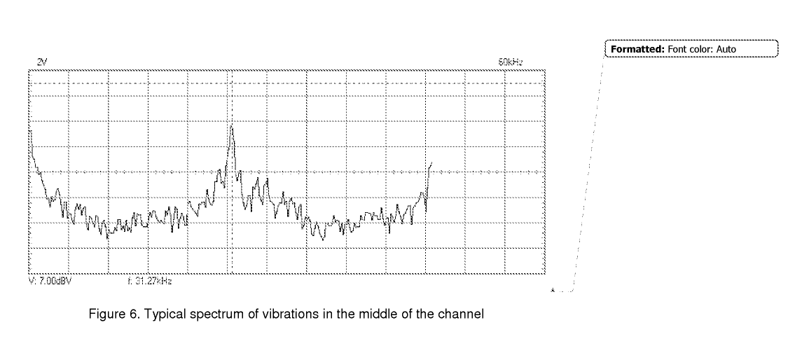

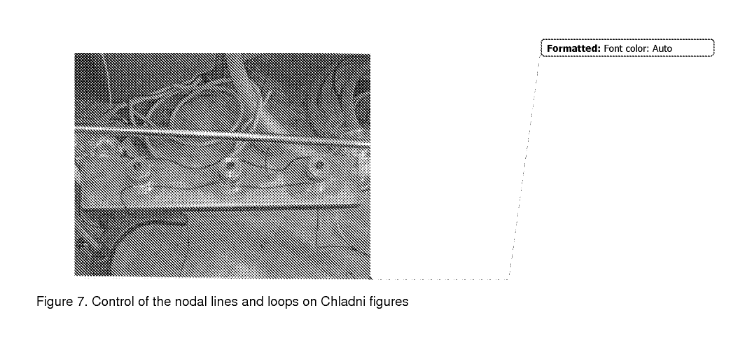

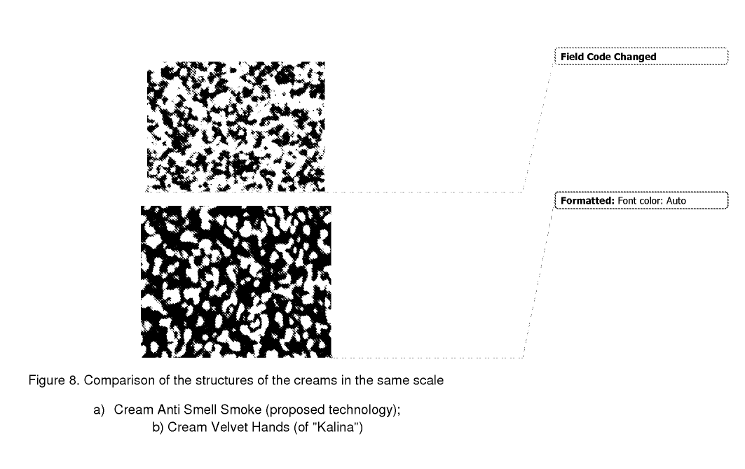

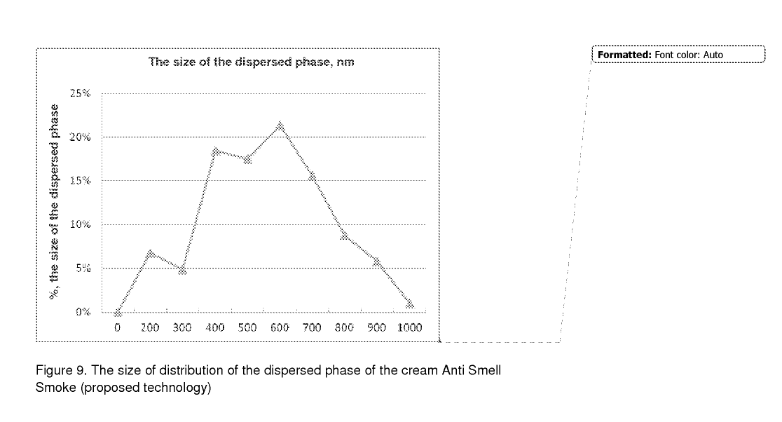

The character of the invention is explained by drawings: FIG. 1 Nutritional cream with healing herbs Mirra (processing by digital filter “smart sharpen”); FIG. 2—Calibration scale, scale division 10 mm; FIG. 3—The size distribution of the dispersed phase of the cream Mirra (prototype). Processing of the photograph shown in FIG. 1.; FIG. 4—The stage of laboratory testing; FIG. 5—The appearance of an industrial plant; FIG. 6—Typical spectrum of vibrations in the middle of the channel; FIG. 7—Control of the nodal lines and loops on Chladni figures; FIG. 8—Comparison of the structures of the creams in the same scale Cream Anti Smell Smoke (proposed technology); b) Cream Velvet Hands (of “Kalina”); FIG. 9—The size of distribution of the dispersed phase of the cream Anti Smell Smoke (proposed technology).

It is known that under certain conditions in any elastic plate of finite size standing waves occur, namely, there are such variations in which all points pulse with the same frequency and the same phase. The types of possible standing waves of the plate depend on its shape. In addition, the form of standing waves depends on the boundary conditions: the edges of the plate can be fixed or can be free. The frequencies of these self-oscillations (i.e., standing waves) depend not only on the size, shape of the plate, the boundary conditions, but also on the speed of wave propagation on the plate, and on a number of other characteristics. For a rectangular plate with fixed edges solution of the wave equation on a set of the frequencies of self-oscillation in the Cartesian coordinate system is given as:

$$ω = c \sqrt{{k}_{x}^{2} + {k}_{y}^{2}} = c \sqrt{{( {j}_{x} \frac{π}{{L}_{x}} )}^{2} + {( {j}_{y} \frac{π}{{L}_{y}} )}^{2}}$$

where c—speed of wave propagation on the plate;

kₓ, k_{y}—wave numbers, the values of which are determined by the boundary conditions;

Lₓ, —length of the side of the plate directed along the axis Ox;

L_{y}—length of the side of the plate directed along the axis Oy;

jₓ, j_{y}—an integer equal to the number of crests of the wave along the respective sides of the plate.

For generating a quasi-plane wave in the gap between the channel walls, it is required to satisfy the conditions of the first fundamental mode when jx, jy are 1. Figure FIG. 4 represents one of the stages of laboratory tests to determine the self-frequencies of the channel, which further is supplied to complete the industrial unit FIG. 5. For the measurements a measuring circuit is used, that passed a test in ROSTEST, the measuring circuit consists of piezo-acceleration transducer of 4344 type and the amplifier 2635 produced by the company Bruel & Kjaer, digital oscilloscope Velleman having function of the fast Fourier transforming with the registration of signals to a personal computer. A linear scale with a high resolution of 60 kHz is applied.

For initiating the ultrasonic vibrations a modified generator UZG 2-22 type with the auto-resonant frequency is used.

In FIG. 6 there is a typical spectrum of vibrations of rectangular shape channel with the dimensions of 0.48 m and 0.1 m, the width of the gap 0.024 m. The calculated value of the fundamental mode to the above formula, is 29.7 kHz (with the velocity of longitudinal waves in a stainless steel˜5800 m/c), the experimentally observed value is 31.27 kHz. Checking of the self-frequency of the fundamental mode must be always be carried out because in practice channels contain welded process connecting shafts for flushing and the theoretical calculation of the frequency channels is difficult.

An additional check is carried out by registering Chladni figures on the surface of the channel. The Q-factor of the channel is 4-6, which substantially improves the energy of emulsification process and eliminates the effect of other harmonics, which were previously observed.

The speed of sound in multi-phase media, such as cosmetic emulsions is not very different from the speed of sound value for water (˜1500 m/s), which allows, with sufficient accuracy for practical purposes, to calculate the optimum clearance h for the channel at a given resonance frequency. The wavelength is about 4.8 cm, respectively, for the superposition of waves within the channel; for a half-wave resonator the width should be˜2.4 cm

The composition of cream Anti Smell Smoke is presented in the Table

| | Content | TOTAL QUANTITY, kg |
|---|---|---|
| Name | % | Phase(temperature) |
| DC 9045 | 0.70 | Active supplement |
| | | 50 |
| DC 345 | 3.00 | Active supplement |
| | | 50 |
| Lanette O | 2.20 | fat |
| Trylon B | 0.05 | water |
| Urea | 3.00 | water |
| Glycerin | 3.00 | water |
| Methylparaben | 0.30 | water |
| Propylparaben | 0.10 | water |
| Kathon CG | 0.05 | Active supplement |
| | | 50 |
| Soybean oil | 0.30 | 50 degrees |
| Flavor | | |
| Cetiol CC | 2.00 | fat |
| Skvalan | 0.50 | fat |
| Emulgade SE-PF | 1.20 | fat |
| edenor | 0.70 | fat |
| DC 200/100 | 1.00 | Active supplement |
| | | 50 |
| Grindox 109 | 0.05 | Active supplement |
| | | 50 |
| DC 200/100 | 1.00 | Active supplement |
| | | 50 |
| Grindox 109 | 0.05 | Active supplement |
| | | 50 |
| Biolin/P | 1.00 | Active supplement |
| | | 50 |
| TegoSorb50 | 1.00 | fat |
| Mint gorophyt | 0.01 | fat |
| Crodamol ML | 0.70 | fat |
| Water | 79.14 | |
| TOTAL | 100.00 | |
| Fat | 8.41 | |
| Watering | 6.35 | |
| Active + flavor | 6.10 | |
| water | 79.14 | |
| TOTAL | 100.00 | |

While preparation of the emulsion there are three stages:

1) Heating of oil phase and water with soluble components and their mixing (production of the base);
2) The beginning of a homogenization and gradual cooling of the base;
3) Adding of the active ingredients and fragrances at a temperature of about 45 degrees (Celsius) and homogenization at the constant temperature.

In stages 2 and 3, respectively, there were given different vibration amplitudes; cavitation control mode was provided by using temperature measurement at the inlet and outlet of the flow channel and according to the indicator of the power supplied to the piezo-oscillators.

In FIG. 8 there is a photo showing “in the same scale a comparison of cream Anti Smell Smoke and cream Velvet hands produced by “Kalina”.

In case of the size of the dispersed phase in the range of 400-700 nanometers the level of homogeneity amounts up 50-60%, that proves the achieving of the required result goal.

This structure of cosmetic emulsions for consumers has several new characteristics:

The tactile and organoleptic characteristics (easy application and grinding, quick absorbency, the absence of a greasy film after application) are significantly improved. Market research (panels) were conducted, in which an anonymous survey showed 87% of women and girls (65 respondents) selected the cream manufactured by the proposed method in comparison with the cream of the same composition, but prepared by traditional technology as the “best” according to organoleptic;

To obtain a stable emulsion it is sufficient to use only 30-40% of an emulsifier of the amount required for the preparation by traditional technology.

It is known that reduction of the emulsifier reduces the risk of destruction of the lipid barrier of the skin, i.e. contributes to maintaining healthy skin;

The obtained emulsion has a pronounced rapid action. With continued use in as little as 5-7 days skin dryness disappeared, even when washing dishes by usual cleaning products, as it is indicated by the respondents.

Experiments show that the proposed method of manufacturing cosmetic emulsion allows to:
obtain a smaller size of the dispersed oil (fat) phase;
obtain a high level of homogeneity of the emulsion.

1) (canceled)

2) A method of producing a cosmetics emulsion from a liquid base and a plurality of components comprising the steps of:
mixing the liquid base and some of the plurality of components comprising the steps of:
heating an oil and water of the liquid base;
adding the components;
agitating the mixture;
homogenizing the mixture, wherein the step of homogenizing comprises:
cooling the mixture;
passing the mixture through a flow channel having a substantially rectangular flow region;
vibrating a first wall of the flow channel at a harmonic of a fundamental frequency of the first wall, thereby forming a standing wave on the first wall;
transferring the vibrations, by at least a portion of the first wall, to the mixture within the flow channel;
vibrating a second wall of the flow channel at a harmonic of a fundamental frequency of the second wall, thereby forming a standing wave on the second wall;
transferring the vibrations, by at least a portion of the second wall, to the mixture within the flow channel;
selecting a vibrational amplitude of the standing wave on the first wall to be above a threshold of acoustic cavitation for the mixture;
selecting a vibrational amplitude of the standing wave on the second wall to be above a threshold of acoustic cavitation for the mixture;
selecting the frequencies of the first wall and the second wall such that their vibrations transferred to the mixture cause the mixture to generate quasi-plane standing waves within the flow channel;
selecting a gap of the flow channel between the first wall and the second wall to have a width that is a multiple of quarter wavelengths of at least one of the first wall and the second wall; and
adding a remainder of the plurality of components, the remainder of the components comprising at least one of active ingredients and fragrance, the step of adding the remainder of the plurality of components being performed while the mixture remains in the flow channel and under vibration from the first wall and the second wall.

3) The method of claim 1 further comprising the step of adjusting a flow rate of the mixture through the flow channel to maintain a steady flow rate.

4) The method of claim 1 wherein the step of adding the components comprises adding an emulsifier, biologically active substances, and solid powdered ingredients to a vegetable oil and water base.

5) The method of claim 1 wherein the step of vibrating a second wall of the flow channel at a harmonic of a fundamental frequency of the second wall further comprises selecting the frequency of the second wall to match the frequency of the first wall.

6) The method of claim 1 wherein the step of vibrating a second wall of the flow channel at a harmonic of a fundamental frequency of the second wall further comprises selecting the frequency of the second wall to be different from the frequency of the first wall.

7) The method of claim 1 further comprising the step of checking a self-frequency of the fundamental mode of the first wall and the second wall.

8) The method of claim 7 further comprising the step of registering a Chlandi figure of a surface of the channel.

9) The method of claim 1 wherein the step of selecting the vibrational amplitude of the first and second wall and the step of selecting the frequencies of the first and second wall are selected to achieve a 50-60% homogeneity of the mixture.
